(19) United States
(12) Patent Application Publication
(10) Pub. No.: US 2026/0272793 A1
(43) Pub. Date: Sep. 17, 2026

(54) PROCESS FOR PREPARING A UV FILTER DISPERSION FOR WET MILLING

(71) Applicant: BASF SE, Ludwigshafen am Rhein (DE)

(72) Inventors: Florian VOGT, Grenzach-Wyhlen (DE); Veronique KESSLER, Grenzach-Wyhlen (DE); Ullrich MENGE, Grenzach-Wyhlen (DE)

(21) Appl. No.: 19/166,965

(22) PCT Filed: Mar. 20, 2024
(86) PCT No.: PCT/EP2024/057460

§ 371 Date: Sep. 19, 2025

(30) Foreign Application Priority Data
Mar. 22, 2023 (EP) 23163532.7

(51) Int. Cl. A61K 8/04; A61K 8/49; A61K 8/60; A61Q 17/04

(52) U.S. Cl. CPC A61K 8/04; A61K 8/496; A61K 8/4966; A61K 8/602; A61Q 17/04

(57) ABSTRACT

A process for producing an aqueous dispersion of a water-insoluble, solid organic UV filter compound, comprising the steps of providing water, a dispersant, and a powder comprising the water insoluble, solid organic UV filter compound; providing an aqueous dispersant solution by adding the dispersant to the water in an amount of 0.001 to 0.25 wt.-% with respect to the total weight of the aqueous dispersant solution leaving a remaining amount of the dispersant; dispersing, using an inline disperser, the powder comprising the water insoluble, solid organic UV filter compound in the aqueous dispersant solution yielding a first aqueous dispersion; adding the remaining amount of the dispersant to the first aqueous dispersion yielding a second aqueous dispersion; further dispersing the second aqueous dispersion yielding the aqueous dispersion of a water-insoluble, solid organic UV filter compound, wherein the total amount of dispersant is higher than 0.25 wt.-% with respect to the total weight of the aqueous dispersant solution.

### TECHNICAL FIELD

This invention relates to a process to produce suspensions of insoluble organic UV absorbers in water using an inline disperser. Foaming is reduced by optimization of the dispersant level and the dosing process in the mixing device. The process is economic and reduces EHS measures in the plant. The suspensions obtained are suitable for wet milling in a stirred media mill to obtain micronized UV Filters for use in cosmetic sunscreens.

### BACKGROUND

Micronized organic UV absorber dispersions are well described in the literature. Suitable organic particulate UV absorbers for use in cosmetic sunscreens are described e.g., in WO 95/22959 A1, WO 97/03643 A1, WO 2009/077356 A1, and WO 2015/155158 A1.

Generally, some of the UV absorbers usually used are insoluble in water. For the production of products using such water-insoluble UV absorbers, it is however necessary to disperse them in water as fine as possible. Thus, the process of dispersing such UV absorbers has been constantly topic of improvements during the recent years.

In particular, the use of dispersants has been topic of research over the years. WO 97/03643 A1 provides a method for producing a composition of a micronized organic UV absorber, which comprises grinding the UV absorber in the presence of an alkyl polyglucoside. More grinding aids are given in WO 2009/068469 A1.

Hence, slurry formation is a crucial step in the large-scale production of micronized organic UV filter dispersions. As stated above, particulate UV absorbers are water-insoluble, crystalline materials and obtained as low-density powders which are dust explosive. Safety measures must be in place when such powders are introduced into the vessel containing the liquid phase.

One problem during dispersing of said UV filters is foam formation. As the powder density is lower than true density, the powder contains air. The wetting of the UV filter powder with the liquid releases the air in the bulk phase and, thus, generates foam. Furthermore, the usage of dispersants, especially surfactants, also promotes foam formation. Foaming occurs also when process or equipment used for mixing and homogenization of the suspension is not adequate. Foam is prohibitive to any stable and robust wet milling process on a stirred media mill. Hence, it has been a topic to prevent foam formation in the process of dispersing said UV filters.

In WO 2009/003934 A1, the dispersions are prepared by grinding the UV filter in an apparatus comprising yttrium-stabilized zirconium oxide grinding beads in the presence of an antifoam agent. WO 2017/198806 A1 describes the wet milling of organic UV filter suspensions in more detail. A formulation comprising water, dispersant and antifoam is prepared, and the UV filter powder is added to form a slurry. This slurry is pre-grinded using a colloid mill, and then fine-grinded in a stirred media mill to mean particle size of d50 from 100 to 170 nm.

However, the usage of antifoam makes it part of the product formulation which is not always acceptable for customers.

WO 2018/069200 A1 provides a wet milling process in a stirred media mill without using antifoam. The UV filter suspension is prepared in a vessel by slowly adding the UV filter powder to the water/alkyl polyglucoside mixture. The suspension is degassed under gentle stirring in a heating/cooling cycle before the milling process starts. It is claimed that by selecting a particular particle size in the UV filter suspension, the foaming during wet milling is controllable. Further reduction of foaming is achieved by using a specific alkyl polyglucoside as the dispersant.

However, a degassing step before wet milling as described in in WO 2018/069200 A1 is expensive and time-consuming, especially in production scale.

### SUMMARY OF THE INVENTION

Thus, the object of the present invention is to provide a scalable process for the dispersion of a water-insoluble, solid organic UV filter in an aqueous phase without adding antifoam agents to the aqueous phase and without degassing steps in the process.

It has now been surprisingly found that this object is solved by a process in which an aqueous dispersant solution is provided, wherein the amount of dispersant is in a range of 0.001 to 0.25 wt.-% with respect to the total weight of the aqueous dispersant solution, subsequently the UV filter powder is dispersed in this aqueous dispersant solution yielding a first aqueous dispersion, and after this dispersion the remaining dispersant is added. This concentration range relates to the active level of the dispersant in the liquid prior to powder addition.

The advantageous technical effect of the present invention is that an aqueous UV filter dispersion can be prepared with significantly reduced or even completely suppressed foam formation, thereby removing the need for any further degassing steps resulting in a fast and efficient way of wetting a water-insoluble, solid organic UV filter.

### BRIEF DESCRIPTION OF THE DRAWINGS

FIG. 1 is a schematic drawing of the device setup as used in the examples CE1, CE2, IE1, and IE2.

FIG. 2 is a picture of the aqueous phase and foam produced in the Comparative Example CE1.

FIG. 3 is a picture of the aqueous phase and foam produced in the Comparative Example CE1.

FIG. 4 is a picture of the number of particles having a diameter of 500 μm or more (left handed sieves) and a diameter in the range of from 200 to 500 μm (right handed sieves) for probes A-D of Comparative Example CE2.

### DEFINITIONS

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%. It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only. Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below. It is to be understood that this invention is not limited to the particular methodology, protocols, reagents etc. described herein as these may vary. It is also to be understood that the terminology used herein is for the purpose of describing particular embodiments only and is not intended to limit the scope of the present invention that will be limited only by the appended claims. Unless defined otherwise, all technical and scientific terms used herein have the same meanings as commonly understood by one of ordinary skill in the art.

As used herein the term “does not comprise” or “free of” means in the context that the composition of the present invention is free of a specific compound or group of compounds, which may be combined under a collective term, that the composition does not comprise said compound or group of compounds in an amount of more than 0.8% by weight, based on the total weight of the composition. Furthermore, it is preferred that the composition according to the present invention does not comprise said compounds or group of compounds in an amount of more than 0.5% by weight, preferably the composition does not comprise said compounds or group of compounds at all.

When referring to compositions and the weight percent of the therein comprised ingredients it is to be understood that according to the present invention the overall amount of ingredients does not exceed 100% (±1% due to rounding).

The term “sunscreen composition” or “sunscreen” refers to any topical product, which absorbs, and which may further reflect and scatter certain parts of UV radiation. Thus, the term “sunscreen composition” is to be understood as not only including sunscreen compositions, but also any cosmetic compositions that provide UV protection. The term “topical product” refers to a product that is applied to the skin and can refer, e.g., to sprays, lotions, creams, oils, foams, powders, or gels. According to the present invention the sunscreen composition may comprise one or more active agents, e.g., organic and inorganic UV filters, as well as other ingredients or additives, e.g., emulsifiers, emollients, viscosity regulators, stabilizers, preservatives, or fragrances.

The term “daily care composition” refers to any topical product, which absorbs, and which may further reflect and scatter certain parts of UV radiation and is used as an everyday care product for the human body, e.g. for face or body. The daily care composition may comprise one or more active agents, e.g., organic and/or inorganic UV filters, as well as other ingredients or additives, e.g., emulsifiers, emollients, viscosity regulators, stabilizers, preservatives, or fragrances. Suitable daily care composition are according to the present invention, e.g. leave-on face and body care products.

Suitable leave-on products for face and body are, e.g., sunscreen compositions, decorative preparations, and skin care preparations.

Suitable decorative preparations are, e.g., lipsticks, nail varnishes, eye shadows, mascaras, dry and moist make-up, rouge, powders, depilatory agents and suntan lotions.

Suitable skin care preparations are e.g., moisturizing, refining, and lifting preparations. The cited daily care compositions can be in the form of creams, ointments, pastes, foams, gels, lotions, powders, make-ups, sprays, sticks or aerosols. The daily-care compositions are therapeutic daily-care compositions since they comprise UV filters.

The term “UV filter” or “ultraviolet filter” as used herein refers to organic or inorganic compounds, which can absorb and may further reflect, and scatter UV radiation caused by sunlight. UV-filter can be classified based on their UV protection curve as UV-A, UV-B, or broadband filters. Preferably, the term “UV filter” comprises or consists of any UV filter as defined in the Annex VI (version of 03.12.2020) of the Regulation (EC) No 1223/2009 of the European Parliament and of the Council.

Water soluble UV filters have a solubility in water of at least 2% by weight, preferably at least 3% by weight, more preferably at least 5% by weight.

The prefix Cn-Cm indicates in each case the possible number of carbon atoms in the group.

The term “C₁₂-C₁₅ alkyl benzoate” refers to esters of benzoic acid with fatty alcohols containing a C₁₂-C₁₅-alkyl chain. C₁₂-C₁₅ alkyl chain is defined as an alkyl chain with C₁₂, C₁₃, C₁₄ or C₁₅ chain length.

The term “Cn-Cm carboxylic acids” as used herein denotes in each case a linear or branched carboxylic acids having from n to m carbon atoms, such as 6 to 24 carbon atoms.

The term “Cn-Cm alcohols” as used herein denotes in each case a linear or branched alcohol having from n to m carbon atoms, such as having from 3 to 24 carbon atoms, or from 6 to 24 carbon atoms, or from 1 to 22 carbon atoms.

The term “C₂-C₁₂ dicarboxylic acids” as used herein denotes in each case a dicarboxylic acid having from 2 to 12 carbon atoms such as butanedioic acid (succinic acid), pentanedioic acid (glutaric acid), hexanedioic acid (adipic acid), or decanedioic acid (sebacic acid).

The term “dialkyl ether” as used herein denotes in each case a linear or branched dialkyl ether having a total of from 12 to 36 carbon atoms and comprising at least one ether moiety.

The term “C₆-C₂₂ alcohol carbonates” as used herein denotes in each case a linear or branched alcohol carbonates having 6 to 22 carbon atoms and comprising at least one functional group consisting of a carbonyl group flanked by two alkoxy groups.

The term “alkyl” as used herein denotes in each case a straight-chain or branched alkyl group having exemplarily from 1 to 18 carbon atoms. Examples of an alkyl group are methyl, ethyl, n-propyl, iso-propyl, n-butyl, 2-butyl, iso-butyl, tert-butyl, n-pentyl, 1-methylbutyl, 2 methylbutyl, 3 methylbutyl, 2,2-di-methylpropyl, 1 ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethyl-butyl, 1,2-dimethylbutyl, 1,3-dimethyl-butyl, 2,2-dimethylbutyl, 2,3-dimethylbutyl, 3,3-dimethyl-butyl, 1-ethylbutyl, 2-ethylbutyl, 1,1,2-trimethylpropyl, 1,2,2-trimethylpropyl, 1-ethyl-1-methyl-propyl, and 1-ethyl-2-methylpropyl.

The term “alkoxy” as used herein denotes in each case a linear or branched alkyl group which is bonded via an oxygen atom and has usually from 1 to 20 carbon atoms. Examples of an alkoxy group are methoxy, ethoxy, n-propoxy, iso-propoxy, n-butyloxy, 2-butyloxy, iso-butyloxy, tert.-butyloxy, and the like.

The term “carboxyalkyl” as used herein includes carboxymethyl, carboxyethyl, carboxypropyl, carboxyisopropyl, carboxybutyl, carboxyisobutyl, carboxyamyl, carboxyhexyl, carboxyheptyl, carboxyoctyl, carboxyisooctyl, carboxynonyl, carboxydecyl, carboxyundecyl, carboxydodecyl, carboxytetradecyl, carboxyhexadecyl, and carboxyoctadecyl, carboxymethyl being preferred.

The term “cycloalkyl” as used herein denotes in each case a monocyclic cycloaliphatic radical having usually from 3 to 10 or from 5 to 8 carbon atoms, such as cyclopropyl, cyclobutyl, cyclopentyl, cyclohexyl, cycloheptyl, cyclooctyl, cyclononyl and cyclodecyl or cyclopropyl, cyclobutyl, cyclopentyl and cyclohexyl.

The term “substituted” as used herein, means that a hydrogen atom bonded to a designated atom is replaced with a specified substituent, provided that the substitution results in a stable or chemically feasible compound. Unless otherwise indicated, a substituted atom may have one or more substituents and each substituent is independently selected.

The term “insoluble, solid organic UV Filter” refers to a plurality of particles of one UV filter with a specific size distribution of the particles.

The size of the UV filter particles in the present invention is defined by the particle size distribution of a set of particles, which may be characterized with respect to particle volume (mass). Volume based distributions may be obtained by laser diffraction, and many commercial instruments are available, for example from Anton Paar (PSA series), Microtrac MRB (Sync) or Malvern Panalytical (Mastersizer series). Depending on sensitivity and resolution of the selected instrument, the numerical results characterizing the particle size distribution will differ within some minor range. The skilled expert knows how to deal with these deviations, and such instruments are routinely used in R&D and quality control labs.

The term “dispersion” as used herein refers to a system in which distributed particles of one material are dispersed in a continuous phase of another material. The two phases may be in the same or different states of matter. A specific subtype of a dispersion is the “suspension”, wherein solid parts are dispersed (i.e., not dissolved) in a fluid.

### DESCRIPTION OF THE INVENTION

The present invention provides a process for producing an aqueous dispersion of a water-insoluble, solid organic UV filter compound, comprising the steps of:
a) Providing water, preferably distilled water, most preferably bidistilled water, a dispersant, and a powder comprising the water insoluble, solid organic UV filter compound;
b) Providing an aqueous dispersant solution by adding the dispersant to the water in an amount of 0.001 to 0.25 wt.-% with respect to the total weight of the aqueous dispersant solution leaving a remaining amount of the dispersant;
c) Dispersing, using an inline disperser, the powder comprising the water insoluble, solid organic UV filter compound in the aqueous dispersant solution yielding a first aqueous dispersion;
d) Adding the remaining amount of the dispersant to the first aqueous dispersion yielding a second aqueous dispersion;
e) Further dispersing the second aqueous dispersion yielding the aqueous dispersion of a water-insoluble, solid organic UV filter compound wherein the total amount of dispersant is higher than 0.25 wt.-% with respect to the total weight of the aqueous dispersant solution.

The process is in particular controlled by the concentration of dispersant used in the aqueous phase before the dispersion step. Preferably, the process is carried out using recycling. In such a preferred embodiment, the aqueous dispersion is added to step c) again. Hence, preferably, the concentration of dispersant is kept constant in the range of 0.001 to 0.25 wt.-% with respect to the total weight of the aqueous dispersant solution until step c) is finished, i.e., the total amount of water-insoluble, solid organic UV filter has been added, thereby recycling the dispersion. After finalization of step c), the remaining dispersant is added in step d).

Preferably, in step b) the dispersant is added in an amount of less than 0.24 wt.-% with respect to the total weight of the aqueous dispersant solution, preferably in an amount of less than 0.2 wt.-%, and most preferably in an amount of less than 0.15 wt.-%. If the dispersant is added in an amount of less than 0.24 wt.-%, the foaming is significantly reduced. If the dispersant is added in an amount of less than 0.15 wt.-%, the foaming is suppressed.

Suitable dispersants are in particular polyglycerol alkyl ester, preferably polyglycerol monoalkyl ester, and alkyl polyglucoside, preferably having the formula

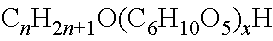

in which n is an integer ranging from 8 to 16, and
x is the mean polymerization level of the glucoside moiety (C₆H₁₀O) and ranges from 1.4 to 1.6,
or an ester thereof. Further suitable dispersing agents are disclosed in WO 2009/7068469 A1.

According to the present invention, the polyglycerol monoalkyl ester has preferably a mean degree of polymerization of glycerol of 5 or more.

In one preferred embodiment, the at least one polyglycerol monoalkyl ester is selected from the group consisting of decaglyceryl caprate, decaglyceryl monolaurate, decaglyceryl myristate, decaglyceryl oleate, decaglyceryl stearate, decaglyceryl isostearate, hexaglyceryl cap rate, hexaglyceryl laurate, hexaglyceryl myristate, hexaglyceryl oleate, hexaglyceryl stearate, hexaglyceryl isostearate, pentaglyceryl caprate, pentaglyceryl laurate, pentaglyceryl myristate, pentaglyceryl oleate, pentaglyceryl stearate, pentaglyceryl isostearate, and combinations thereof. In a particular preferred embodiment, the at least one polyglycerol monoalkyl ester is decaglyceryl monolaurate (INCI polyglyceryl-10 laurate).

Polyglycerol monoalkyl esters having an HLB (hydrophilic-lipophilic balance) of 14.5 or more are preferable and having an HLB of 15 or more are more preferable. The HLP value is determined by the formula

$$HLB = 20 {M}_{h} / M ,$$

wherein Mₕ is the molecular mass of the hydrophilic portion of the molecule and M is the molecular mass of the whole molecule.

Polyglycerol monoalkyl esters having an HLB of less than 14.5 may take a longer time for dispersion of micronized methylene bis-benzotriazolyl tetramethylbutylphenol in water phase components. Examples of polyglycerol monoalkyl esters with a mean degree of polymerization of 5 or more and having an HLB of 14.5 or more may include decaglyceryl caprate, decaglyceryl monolaurate, decaglyceryl myristate, decaglyceryl oleate, decaglyceryl stearate, decaglyceryl isostearate, hexaglyceryl laurate, pentaglyceryl laurate, pentaglyceryl myristate, pentaglyceryl stearate, and pentaglyceryl oleate, and those having an HLB of 15 or more may include decaglyceryl caprate and decaglyceryl monolaurate.

Particularly preferred are polyglyceryl monolaurate, particularly decaglyceryl monolaurate, and decyl glucoside.

Preferably, the alkyl polyglucoside consists of a C₁-C₁₂ ester of the compound of formula CₙH₂ₙ₊₁O(C₆H₁₀O₅)ₓH, namely an ester formed by reacting a C₁-C₁₂ carboxylic acid with one or more free PH group of the glucoside moiety (C₆H₁₀O). In this connection, it is preferred that the ester is formed by reacting formic, acetic, propionic, butyric, sulfosuccinic, citric, or tartaric acid, with one or more free OH groups on the glucoside moiety (C₆H₁₀O).

Suitable alkyl polyglucoside according to the present invention are the alkyl polyglucosides known under the INCI name ‘decyl glucoside’ [CAS 68515-73-1], such as in particular the C₈₋₁₆ alkyl polyglucoside which is e.g. available as PlantaCare 2000 UP from BASF. Preferably, the dispersant is an alkyl glucoside, more preferably a poly alkyl glucoside, and most preferably the active compound in Plantacare® 2000 UP. It should be understood that the term dispersant as used herein denotes the active tenside compound. Hence, e.g., if the dispersant is added as a solution in water, only the amount of tenside therein is relevant.

The term insoluble, solid organic UV filter refers to UV filters that are not soluble in water and cosmetic oils at 25° C. To the contrary, water-soluble UV filters have a solubility in water of at least 2% by weight, preferably at least 3% by weight, more preferably at least 5% by weight and oil soluble UV filters have a solubility in common cosmetic oils, such as C₁₂-C₁₅-alkyl benzoate, dibutyl adipate, diisopropyl sebacate, phenethyl benzoate, or dicaprylyl carbonate of at least 2% by weight, preferably at least 5% by weight, more preferably at least 7% by weight.

It is preferred that the insoluble, solid organic UV Filter is selected from the group consisting of tris-biphenyl triazine, 1,1′-(1,4-piperazinediyl)bis[1-[2-[4-(diethylamino)-2-hydroxybenzoyl]phenyl]-methanone, phenylene bis-diphenyltriazine, and 2,2′-methylenebis[6-(2H-1,2,3-benzotriazol-2-yl)-4-(2,4,4-trimethylpentan-2-yl)phenol].

Tris-biphenyl triazine has the following structure:

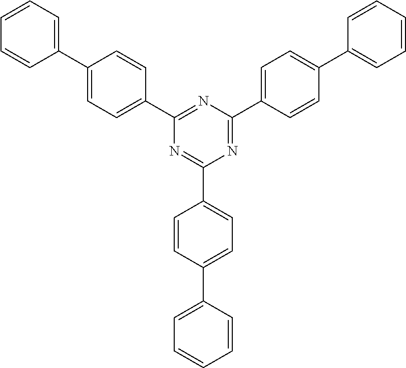 [SMILES: c1ccc(-c2ccc(-c3nc(-c4ccc(-c5ccccc5)cc4)nc(-c4ccc(-c5ccccc5)cc4)n3)cc2)cc1]

1,1′-(1,4-piperazinediyl)bis[1-[2-[4-(diethylamino)-2-hydroxybenzoyl]phenyl]-methanone has the following structure:

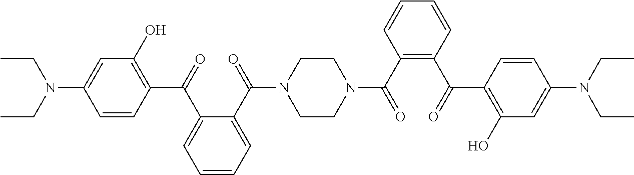 [SMILES: CCN(CC)c1ccc(C(=O)c2ccccc2C(=O)N2CCN(C(=O)c3ccccc3C(=O)c3ccc(N(CC)CC)cc3O)CC2)c(O)c1]

Phenylene bis-diphenyltriazine has the following structure:

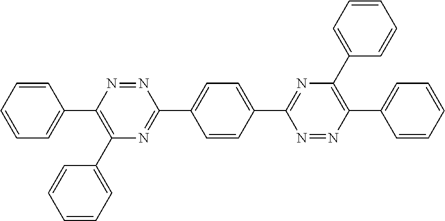 [SMILES: c1ccc(-c2nnc(-c3ccc(-c4nnc(-c5ccccc5)c(-c5ccccc5)n4)cc3)nc2-c2ccccc2)cc1]

2,2′-methylenebis[6-(2H-1,2,3-benzotriazol-2-yl)-4-(2,4,4-trimethylpentan-2-yl)phenol] has the following structure:

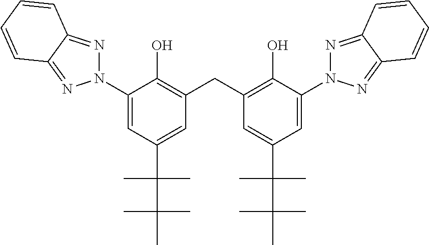 [SMILES: CC(C)(C)C(C)(C)c1cc(Cc2cc(C(C)(C)C(C)(C)C)cc(-n3nc4ccccc4n3)c2O)c(O)c(-n2nc3ccccc3n2)c1]

According to one embodiment of the invention, the insoluble, solid organic UV Filter is 1,1′-(1,4-piperazinediyl)bis[1-[2-[4-(diethylamino)-2-hydroxybenzoyl]phenyl]-methanone.

According to another embodiment of the invention, the insoluble, solid organic UV Filter is tris-biphenyl triazine. Tris-biphenyl triazine (also: TBPT) is a broad-spectrum UV filter that as a high performance over the entire UV-A and UV-B spectrum.

According to another embodiment of the invention, the insoluble, solid organic UV Filter is 2,2′-methylenebis[6-(2H-1,2,3-benzotriazol-2-yl)-4-(2,4,4-trimethylpentan-2-yl)phenol]. 2,2′-methylenebis[6-(2H-1,2,3-benzotriazol-2-yl)-4-(2,4,4-trimethylpentan-2-yl)phenol] is a highly effective broadband UV filter which covers the entire UV-A and UV-B spectrum.

Hence, most preferably, the water-insoluble, solid organic UV filter compound is 2,2′-Methylenebis[6-(2H-1,2,3-benzotriazol-2-yl)-4-(2,4,4-trimethylpentan-2-yl)phenol] (MBBT) or 2,4,6-Tris([1,1′-biphenyl]-4-yl)-1,3,5-triazin (TBPT).

In the process according to the present invention, the powder preferably consists of the water-insoluble, solid organic UV filter compound. Preferably, the powder has a bulk density in the range of from 200 to 800 kg/m³. Further preferably, the powder has an median particle size D50 in the range of from 30-400 μm, wherein the particle size is measured by laser diffraction using a Mastersizer 3000 (Malvern Panalytical) and/or a particle size D90 determined with laser diffraction in the range of from 100 to 1000μ This ensures that the surface of the water-insoluble, solid organic UV filter is high enough to make it dispersable in water. Background information on laser diffraction method and how to determine particle size distribution of UV filter powders can be found in WO 2018/069200 A1.

Step c) is carried out using an inline disperser. Inline dispersers are well-known means for achieving a fast incorporation of powders in liquids, and are commercially available from e.g., IKA GmbH & Co. KG, Ystral GmbH, and Netzsch GmbH & Co. KG. Basically, an inline disperser consists of a mixing chamber which is connected to the powder supply, i.e., a powder fluidization device. The liquid is passing quickly through a mixing chamber and creates a drag. The powder is sucked into the mixing chamber and deaerated to some extent before it comes in contact with the liquid. This enhances the wetting process and reduces the formation of stable foam in the dispersion. Safety measures are reduced, as explosive dust in the vessel is avoided. Thus, preferably in step c) the powder is added under vacuum. Thereby, the term “added under vacuum” as used herein denotes a process step or a device, which produces a pressure, which is lower than the environmental pressure, and thereby a driving force for the powder, preferably fluidized powder, to be added. Such a lower internal pressure is e.g. produced by an inline disperser during operation. The lower internal pressure is used to support the movement of the powder, preferably fluidized powder, from the powder supply into the dispersing chamber of the inline disperser thereby increasing the overall dispersion efficiency of the device and process. Preferably, there is a rapid deaeration of the water-insoluble, solid organic UV Filter powder in the vessel, which is further enhanced by using a high energy mixer/stirrer for slurry homogenization in the vessel. Furthermore, an inline disperser is suitable for continuous processes and can be readily installed in a process.

Hence, the device for carrying out the process of the present invention comprises an inline dispenser, wherein the inlet of the inline dispenser is connected to a supply container, which holds the liquid phase. The line connecting the supply container includes a dosing device for dosing the dispersant into the supply line. This dosing device can comprise a dosing pump and a dosing valve. Finally, the powder inlet of the inline disperser is preferably connected to a powder fluidization device and the dosing of the dispersant will be positioned downstream of the inline disperser.

In a preferred embodiment of the device of the present invention, the outlet of the inline disperser is connected to the supply container. However, in this case the outlet line preferably extends into the supply container in form of a dip pipe. Furthermore, the dosing of the dispersant is positioned upstream of the supply container in such preferred embodiment. The setup of such a preferred embodiment is suitable to carry out the process of the present invention in terms of recyclization, whereby the dispersion formed in the inline disperser is added to the supply container and thus redispersed.

Preferably, the supply container comprises a mixer, most preferably a jet mixer. It has been surprisingly found out that a jet mixer despite the higher mixing rates applied is able to significantly reduce the appearance of bubbles in the supply container if the device is used in recyclization mode, i.e., by connecting the outlet of the inline disperser to the supply container. Thereby, air introduced by the powder in the inline disperser is set free in the supply container. If a jet mixer is not used, large bubbles will form further disturbing the process, reducing efficiency. Hence, a jet mixer improved process efficiency by elimination the formation of large air bubbles.

Hence, the process of the present invention provides in step c) a preferred addition speed of the powder in the range of from 20 to 200 kg/min, more preferably in the range of from 100 to 150 kg/min, and most preferably in the range of from 130 to 140 kg/min. Preferably, in step c), the powder is a fluidized powder. Thus, the process of the present invention is significantly faster than the process including degassing steps, such as described e.g., in WO 2018/069200 A1.

Preferably, the inline disperser comprises a high-shear mixer. Even more preferably, in step c) the high-shear mixer is operated at a rotating speed in the range of from 2000 to 4000 rpm, preferably 2500 to 3600 rpm, and most preferably of 2900 to 3100 rpm. This range has been found to achieve the best results in view of process step c). This range has the effect of bringing the water-insoluble, solid organic UV filter into dispersion even at the low levels of dispersant as provided in step b).

Preferably, step c) and step e) are carried out in similar devices or even the same device. Hence, preferably, step e) is carried out using an inline disperser, preferably an inline disperser comprising a high-shear mixer. Even more preferably, in step e) the high-shear mixer is operated at a rotating speed in the range of from 2500 to 4500 rpm, preferably 3000 to 4000 rpm, and most preferably of 3500 to 3700 rpm.

Most preferably, steps c) and e) are carried out using the same inline disperser, preferably inline disperser comprising a high-shear mixer. This simplifies the setup used for carrying out the process. Such a setup is advantageous if the process is carried out as a recyclization process.

As set out above, preferably, step c) is carried out using a dosing pump. Steps d) and e) can be carried out in parallel. This renders the process even more efficient. Moreover, the dispersant is evenly distributed in the aqueous phase avoiding unwanted concentration effects. Preferably during steps a) and b) no dispersing is carried out.

Preferably, the process of the present invention is carried out at a temperature in the range of from 15 to 45° C., more preferably 20 to 30° C.

Preferably, the weight ratio of the water to the water-insoluble, solid organic UV filter compound is in the range of from 0.4 to 1.0, more preferably in the range of from 0.6 to 0.8, and most preferably in the range of from 0.65 to 0.75. Also preferably, after step d) the weight ratio of the dispersant to the water-insoluble, solid organic UV filter compound is in the range of from 0.4 to 1.0, more preferably in the range of from 0.6 to 0.8, and most preferably in the range of from 0.65 to 0.75. These ratios ensure that foam formation is reduced, and the viscosity increase of the dispersion is tolerable for the inline disperser.

Preferably, the process according to the present invention is carried out without the addition of an antifoam agent. Hence, preferably, during the process according to the present invention in the first aqueous dispersion, in the second aqueous dispersion, and/or in the aqueous dispersion of a water-insoluble, solid organic UV filter compound of the process according to the present invention no antifoam is present. More preferably, during the process according to the present invention in the first aqueous dispersion, in the second aqueous dispersion, and in the aqueous dispersion of a water-insoluble, solid organic UV filter compound of the process according to the present invention no antifoam is present.

The term “antifoam agent” as used herein denotes a compound, which is capable of reducing or suppressing at least partially f the formation of foam during a dispersion process.

The dispersion produced by the process of the present invention preferably is suitable for being further processed, i.e., in the area of sunscreen production. For such further processing, certain properties of the dispersion are advantageous. Hence, preferably, the aqueous dispersion of a water-insoluble, solid organic UV filter compound has an average particle size D50 in the range of from 50 to 100 μm, more preferably in the range of from 60 to 80 μm, and most preferably in the range of from 68 to 75 μm. Likewise, preferably, the aqueous dispersion of a water-insoluble, solid organic UV filter compound has a particle size distribution D90/D10 in the range of from 50 to 125, more preferably in the range of from 60 to 120, and most preferably in the range of from 70 to 110. Not only for the further processing, but also to ensure best results in the transport of the slurry in the process of the invention, the density of the dispersant in the aqueous dispersion of a water-insoluble, solid organic UV filter compound is preferably in the range of from 800 to 1400 g/l, more preferably in the range of from 900 to 1300 g/l, and most preferably in the range of from 1000 to 1200 g/l.

Preferably, the aqueous dispersant solution obtained in step b) comprises the dispersant in an amount of 0.001 to 0.24 wt.-%, preferably 0.01 to 0.2 wt.-%, and most preferably 0.1 to 0.15 wt.-%, based on the total weight of the aqueous dispersion.

Preferably, the first aqueous dispersion obtained in step c) comprises the dispersant in an amount of 0.001 to 0.2 wt.-%, preferably 0.01 to 0.1 wt.-%, and most preferably 0.05 to 0.07 wt.-%, based on the total weight of the aqueous dispersion.

Preferably, the first aqueous dispersion obtained in step c) comprises the water-insoluble, solid organic UV Filter in an amount of 35 to 80 wt.-%, preferably 40 to 75 wt.-%, and most preferably 50 to 70 wt.-%, based on the total weight of the aqueous dispersion.

The weight ratio of the dispersant to water-insoluble, solid organic UV Filter in the first aqueous dispersion obtained in step c is preferably from 0.00001 to 0.1, more preferably from 0.0001 to 0.0023, even more preferably from 0.0001 to 0.0020, and in particular from 0.0005 to 0.0015.

Preferably, the aqueous dispersion obtained in step e) comprises the water-insoluble, solid organic UV Filter in an amount of 10 to 65 wt.-%, preferably 35 to 60 wt.-%, and most preferably 50 to 58 wt.-%, based on the total weight of the aqueous dispersion.

Preferably, the aqueous dispersion obtained in step e) comprises the dispersant in an amount of 1 to 50 wt.-%, more preferably of 2 to 30 wt.-%, and in particular of 3 to 6 wt.-%, based on the total weight of the aqueous dispersion.

Preferably, the aqueous dispersion obtained in step e) comprises the water in an amount of 10 to 65 wt.-%, preferably 35 to 45 wt.-%, based on the total weight of the aqueous dispersion.

The weight ratio of the dispersant to water-insoluble, solid organic UV Filter in the aqueous dispersion obtained in step e) is preferably from 0.01 to 0.3, more preferably from 0.05 to 0.25, and in particular from 0.07 to 0.11.

In the aqueous dispersion, the weight ratio of the water to the water-insoluble, solid organic UV filter compound is preferably in the range of from 0.4 to 1.0, more preferably in the range of from 0.6 to 0.8, and most preferably in the range of from 0.74 to 0.79.

The aqueous dispersion may contain further excipients such as colorants, pH adjusters, preservatives which may be incorporated in step b) or added after step e).

Usually, the dispersion is further processed in a colloid mill with subsequent milling in a stirred media mill to achieve the necessary product properties. In case of the process of the present invention, the first step, i.e., the milling in a colloid mill can preferably be skipped, as the formation of foam is suppressed and also the dispersion quality of the dispersion is already high enough to be directly fed to the stirred media mill. Hence, the process of the present invention has further the advantageous effect that processing steps after the process are simplified and made more efficient.

### Examples

### Measurement Methods

### a) Particle Size

The particle size was determined using laser diffraction (Malvern Mastersizer 3000, Fraunhofer model, feed pressure 0.2 bar). Further information on this particle size characterization method can e.g., be found in ‘Particle Characterization: Light Scattering Methods’ by Renliang Xu, Kluwer Academic Publishers (ISBN 0-306-47124-8). If nothing else is stated all particle sizes referring to the nano-sized insoluble organic UV absorber are Dv50 values (volume diameter, 50% of the population resides below this point, and 50% resides above this point) determined by light scattering.

### b) Bulk Density

The bulk density was measured as the apparent density which is measured according to DIN/EN ISO 60. Equipment for bulk density measurements is available e.g., from Landgraf Laborsysteme HLL GmbH, Germany.

### General Experimental Setup

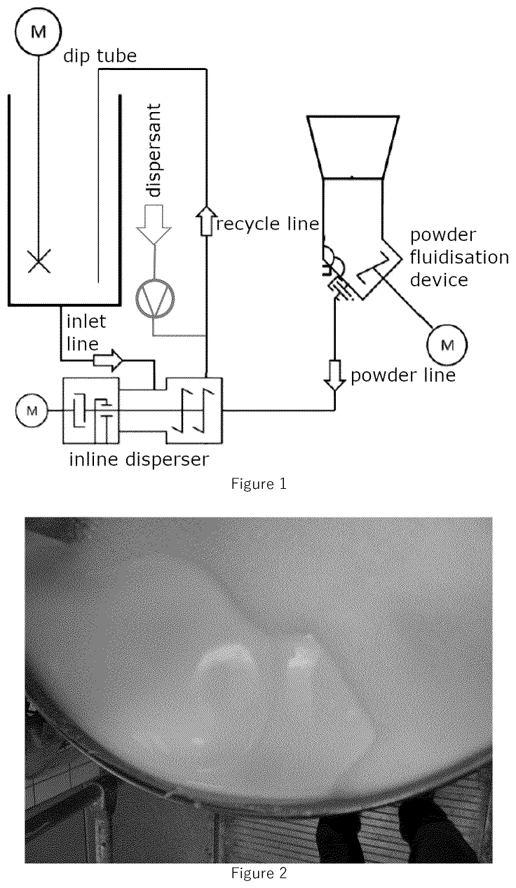

The following description refers to FIG. 1. The inlet of a Conti-TDS 3 inline disperser of Ystral GmbH was connected by an inlet line to a cylindrical container with a volume of 250 L, a height of 127 cm, and a diameter of 50 cm, which was equipped with an electric mixer and a thermometer. The powder inlet of the Conti TDS 3 was connected via a powder line with a powder fluidization device. The outlet of the Conti TDS 3 was equipped with an outlet line, which was again connected with a dip tube reaching to the bottom of the container to achieve recyclization. The inlet line was equipped with a dosage device for dosing dispersant. The dispersant used in all examples was Plantacare® 2000 UP commercially available by BASF SE. This material is an aqueous solution of alkyl polyglucoside and contains about 50% of dispersant. The water-insoluble, solid organic UV filter used in all examples was 2,2′-Methylen-bis-(6-(2H-benzotriazol-2-yl)-4-(1,1,3,3-tetramethylbutyl)phenol) (MBBT) commercially available as Tinosorb M by BASF SE in powdered form. The MBBT material was a powder with a bulk density of about 500-600 g/L.

### Comparative Example CE1

The container was filled with 85 kg bidistilled water having a temperature of 20.9° C. No dispersant was added to the container. The dosage of dispersant was configured to be 20 g/s (i.e., 10 g/s active compound).

The dispersion was started and configured to be performed at 3000 rpm. Within 34 s 120 kg UV filter were added, upon which a heavy foam development could be observed. The temperature in the container increased to 21.7° C. The dosage was also stopped after 34 s. The first aqueous dispersion obtained had a dispersant concentration of 0.165 wt.-% and a UV filter concentration of 58.3 wt.-%.

The dispersion was still further dispersed (i.e., without any further dispersant or UV filter addition) for 60 s at 3000 rpm, resulting in a further increase of the temperature in the container to 23.1° C.

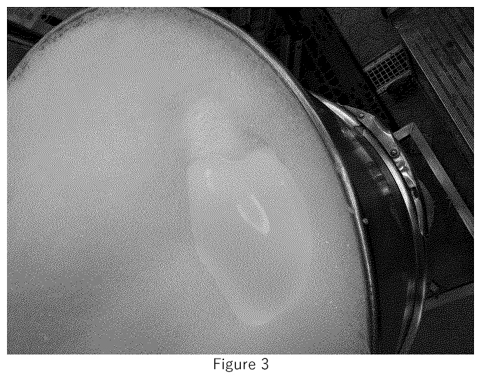

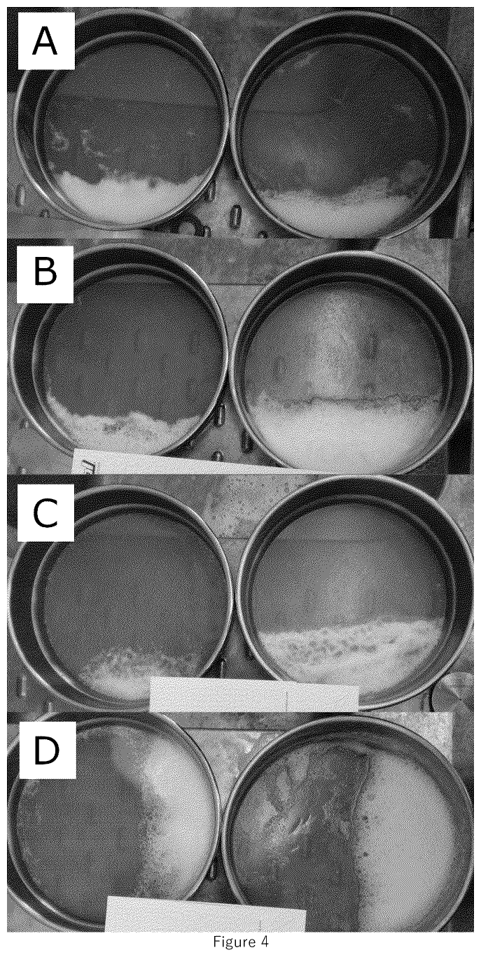

The foam produced had rough foam pores at the surface and creamy, finely dispersed foam in the remaining liquid phase, cf. FIGS. 2 and 3.

Nevertheless, the foam development was too rapid and too strong.

### Comparative Example CE2

The container was filled with 85 kg bidistilled water having a temperature of 21.1° C. The container was filled so that distance of the surface of the liquid to the upper edge of the container was 80 cm. 500 g dispersant (i.e., 250 g active compound) was added to the container: The dosage of dispersant was configured to be off. The aqueous dispersant solution obtained had a dispersant concentration of 0.292 wt.-%.

The dispersion was started and configured to be performed at 3000 rpm. Within 37 s 120 kg UV filter were added, upon which a heavy foam development could be observed. The foam produced had rough foam pores at the surface and creamy, finely dispersed foam in the remaining liquid phase The temperature in the container increased to 21.7° C. The foam volume reached a distance of the surface of the foam to the upper edge of the container of 15 cm.

The dispersion was still further dispersed (i.e., without any further dispersant or UV filter addition) for 60 s at 3000 rpm, resulting in a decrease of the volume of the foam to reach a distance of the surface of the foam to the upper edge of the container of 20 cm.

The dispersion was still further dispersed (i.e., without any further dispersant or UV filter addition) for 60 s at 3000 rpm, resulting in a decrease of the volume of the foam to reach a distance of the surface of the foam to the upper edge of the container of 30 cm. The temperature of the liquid was 26° C.

The remaining amount of UV filter was added within 17 s under further dispersion (3000 rpm), resulting in a maintained volume of the foam of a distance of the surface of the foam to the upper edge of the container of 30 cm.

The dispersion was still further dispersed (i.e. without any further dispersant or UV filter addition) for 50 s at 3000 rpm. The dispersion had to be stopped due to too high density of the liquid, resulting in a decrease of the volume of the foam to reach a distance of the surface of the foam to the upper edge of the container of 35 cm. The temperature of the liquid was 28° C. The first aqueous dispersion obtained had a dispersant concentration of 0.121 wt.-% and a UV filter concentration of 58.5 wt.-%.

Finally, the remaining dispersant (22 kg, i.e., 11 kg active compound) was dosed in and the density of the liquid was instantly reduced, resulting in an increase of the volume of the foam to reach a distance of the surface of the foam to the upper edge of the container of 27 cm. The temperature of the liquid was 28° C. A probe was taken (A).

The liquid was finally dispersed for 1 min at 3000 rpm (Probe B, 29.5° C.), for another 1 min at 3000 rpm (Probe C, 31.2° C.), and for another 1 min at 3600 rpm (Probe D, 33.3° C.).

Nevertheless, as the aim was to disperse 125 kg MBBT per 85 kg bidistilled water, the foam development was too rapid.

### Inventive Example IE1

The container was filled with 85 kg bidistilled water having a temperature of 20.6° C. The container was filled so that distance of the surface of the liquid to the upper edge of the container was 80 cm. 400 g dispersant (i.e., 200 g active compound) was added to the container. The dosage of dispersant was configured to be off. The aqueous dispersant solution obtained had a dispersant concentration of 0.234 wt.-%.

First, the system was deaerated by setting the inline disperser to 600 rpm. The dispersion was started and configured to be performed at 3000 rpm. Within 54 s 125 kg UV filter were added, upon which a certain foam development could be observed. The foam produced had rough foam pores at the surface and creamy, finely dispersed foam in the remaining liquid phase The temperature in the container increased to 21.5° C. The foam volume reached a distance of the surface of the foam to the upper edge of the container of 32 cm maximum and 36 cm after finalization of the dispersion. The first aqueous dispersion obtained had a dispersant concentration of 0.0951 wt.-% and a UV filter concentration of 59.5 wt.-%.

The remaining dispersant (22.1 kg, i.e., 11.05 kg active compound) was dosed in at maximum speed while the inline disperser was configured at 3000 rpm. The temperature of the liquid was 21.8° C. and the distance of the surface of the foam to the upper edge of the container was 27 cm. A probe was taken (E).

The liquid was finally dispersed for 3 min at 3000 rpm (Probe F, 26,4° C., 27 cm distance), and for another 1 min at 3600 rpm (Probe F, 28,6° C., 28 cm distance).

### Inventive Example IE2

The container was filled with 85 kg bidistilled water having a temperature of 20.7° C. The container was filled so that distance of the surface of the liquid to the upper edge of the container was 85.5 cm. 250 g dispersant (i.e., 125 g active compound) was added to the container, which are 1.1 wt.-% of the total weight of dispersant to be added. The dosage of dispersant was configured to be off. The aqueous dispersant solution obtained had a dispersant concentration of 0.146 wt.-%.

First, the system was deaerated by setting the inline disperser to 600 rpm. The dispersion was started and configured to be performed at 3000 rpm. Within 54 s 125 kg UV filter were added, upon which no foam development could be observed, but a small increase in viscosity of the liquid (could still be processed by the inline disperser). The temperature in the container increased to 21.5° C. The liquid volume reached a distance of the surface of the liquid to the upper edge of the container of 36.5 cm. A probe was taken (G). The first aqueous dispersion obtained had a dispersant concentration of 0.0595 wt.-% and a UV filter concentration of 59.5 wt.-%.

The remaining dispersant (22.25 kg, i.e., 11.125 kg active compound) was dosed in at maximum speed while the inline disperser was configured at 3000 rpm (already during the next dispersion step, cf. below), leading to a strong decrease in viscosity of the dispersion. The observed increased viscosity of the liquid instantly disappeared.

The liquid was finally dispersed for 3 min at 3000 rpm (Probe H, 26,9° C., 27 cm distance), and for another 1 min at 3600 rpm (Probe 1, 29° C., 27 cm distance).

### SUMMARY

The comparison of Comparative Example 1 with the remaining examples shows that the addition of the total weight of dispersant necessary to form the respective dispersion results in the rapid and heavy formation of foam making it impossible to further process the liquid/foam system.

As shown by Comparative Example 2, even the reduction to below 0.3 wt.-% dispersant per aqueous dispersant solution resulted in heavy foam formation. At 0.234 wt.-% dispersant per aqueous dispersant solution, the foam production was significantly reduced. At 0.146 wt.-% dispersant per aqueous dispersant solution no foam could be detected anymore.

TABLE 1
Particle sizes

| Probe | D10 [μm] | Median [μm] | D90 [μm] | D90/D10 |
|---|---|---|---|---|
| D | 1.28 | 27.21 | 162.05 | 126.60 |
| A | 1.89 | 68.45 | 288.90 | 152.86 |
| C | 1.62 | 60.72 | 216.00 | 133.33 |
| H | 2.35 | 71.63 | 222.63 | 94.74 |
| I | 2.19 | 71.69 | 208.88 | 95.38 |

1.-15. (canceled)

16. A process for producing an aqueous dispersion of a water-insoluble, solid organic UV filter compound, comprising the steps of:
a) providing water, a dispersant, and a powder comprising the water insoluble, solid organic UV filter compound;
b) providing an aqueous dispersant solution by adding the dispersant to the water in an amount of 0.001 to 0.25 wt.-% with respect to the total weight of the aqueous dispersant solution leaving a remaining amount of the dispersant;
c) dispersing, using an inline disperser, the powder comprising the water insoluble, solid organic UV filter compound in the aqueous dispersant solution yielding a first aqueous dispersion;
d) adding the remaining amount of the dispersant to the first aqueous dispersion yielding a second aqueous dispersion; and
e) further dispersing the second aqueous dispersion yielding the aqueous dispersion of a water-insoluble, solid organic UV filter compound;
wherein the total amount of dispersant is higher than 0.25 wt.-% with respect to the total weight of the aqueous dispersant solution.

17. The process according to claim 16, wherein in step b) the dispersant is added in an amount of less than 0.24 wt.-% with respect to the total weight of the aqueous dispersant solution.

18. The process according to claim 16, wherein the powder consists of the water insoluble, solid organic UV filter compound.

19. The process according to claim 16, wherein the powder is added in step c) at a speed in the range of from 50 to 200 kg/min.

20. The process according to claim 16, wherein the powder of step c) is a fluidized powder.

21. The process according to claim 16, wherein the step c) is carried out using an inline disperser comprising a high-shear mixer.

22. The process according to claim 21, wherein in step c) the high-shear mixer is operated at a rotating speed in the range of from 2000 to 4000 rpm.

23. The process according to claim 16, wherein the step e) is carried out using an inline disperser.

24. The process according to claim 23, wherein the inline disperser comprises a high-shear mixer.

25. The process according to claim 24, wherein in step e) the high-shear mixer is operated at a rotating speed in the range of from 2500 to 4500 rpm.

26. The process according to claim 22, wherein steps c) and e) are carried out in the same high-shear mixer.

27. The process according to claim 16, wherein the step d) is carried out using a dosing pump.

28. The process according to claim 16, wherein in the aqueous dispersion the weight ratio of the water to the water-insoluble, solid organic UV filter compound is in the range of from 0.4 to 1.0.

29. The process according to claim 16, wherein the water-insoluble, solid organic UV filter compound is 2,2′-Methylenebis[6-(2H-1,2,3-benzotriazol-2-yl)-4-(2,4,4-trimethylpentan-2-yl)phenol] (MBBT) or 2,4,6-Tris([1,1′-biphenyl]-4-yl)-1,3,5-triazin (TBPT).

30. The process according to claim 16, wherein the dispersant is an alkyl glucoside having the formula: 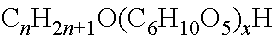
in which
n is an integer ranging from 8 to 16, and
x is the mean polymerization level of the glucoside moiety (C₆H₁₀O) and ranges from 1.4 to 1.6.
